(19) United States
(12) Patent Application Publication
(10) Pub. No.: US 2022/0110712 A1
(43) Pub. Date: Apr. 14, 2022

(54) Surgical Instrument Holder

(71) Applicant: National Central University, Taoyuan City (TW)

(72) Inventors: CHING SHIOW TSENG, Taoyuan City (TW); HUNG-JUNG CHEN, Taoyuan City (TW)

(73) Assignee: National Central University, Taoyuan City (TW)

(21) Appl. No.: 17/074,987

(22) Filed: Oct. 20, 2020

(30) Foreign Application Priority Data
Oct. 8, 2020 (TW) 109135017

(51) Int. Cl. A61B 90/50; A61B 17/00

(52) U.S. Cl. CPC A61B 90/50; A61B 2090/506; A61B 17/00234

(57) ABSTRACT

The present invention relates to a surgical instrument holder. The holder includes an assembly of linkages comprising a first line of center and a second line of center, wherein where the first line of center and the second line of center intersect each other forms a stagnation point; a securing module configured to secure a surgical instrument and define a first line of center; and a complex lockable joint configured to define a second line of center, and comprising a turn piece to selectively lock up the assembly of linkages.

### CROSS-REFERENCE TO RELATED APPLICATION

This application claims priority benefit to Taiwan Invention Patent Application Serial No. 109135017, filed on Oct. 8, 2020, in Taiwan Intellectual Property Office, the entire disclosures of which are incorporated by reference herein.

### FIELD

The present invention relates to a surgical instrument holder, in particular to a surgical instrument holder for a minimally invasive surgery.

### BACKGROUND

During implementation of the minimally invasive surgery, a small incision is made on a patient's body as an entry point and then a surgical instrument is inserted into the patient's body through the entry point, and then a surgeon operates the surgical instrument to perform surgery according to the preoperative plan. In recent years, the minimally invasive surgery is usually performed with an endoscopy, and the endoscope is also used to carry necessary surgical instruments into the patient's body through the entry point, and the endoscopy can achieve the purpose of viewing the internal organs or tissues of the human body with the least damage, so that the surgeon can operate the surgical instruments to safer and more accurately perform operation according to images returned form the endoscopy.

The conventional endoscopy looks like a slender hose or straight tube, and mainly includes a lighting system, image transmission lines (including lenses and optical fibers), an endoscopy body, a working channel, and other surgical instrument such as a scissor, a clamp, hemostat, or electrode. The working principle of the endoscopy is that the lighting system lightens the space inside the body, the lens or camera captures the images of the internal tissues or organs of the human body, and the captured images are transmitted back to the eyepiece or a display through the optical fibers. The endoscopy can serve as the doctor's other eye, and an endoscope holder is required to fasten the endoscopy and related surgical instruments for operations during the minimally invasive surgery.

The main structure of the conventional endoscopy holder generally includes a linkage mechanism having an end configured to fix with the base and other end configured to mount with a clamp, and the clamp is used to clamp the hose of the endoscope, so as to fasten the endoscopy. For example, Patent Cooperation Treaty (PCT) Invention Patent Application publication No. WO/2016/157571 “Endoscope Holding Device” discloses an endoscopy holding device with a multi-linkage structure; the multi-linkage structure is used to maintain precise balance between the endoscopy and the counterweight, so that the endoscopy can be maintained at any position desired by the surgeon and assistant and can be moved as expected by only a small amount of force, thereby achieving efficient surgery.

The U.S. Pat. No. 10,568,493B2 “Medical Instrument and Method for Pivoting Such a Medical Instrument” discloses an endoscopy holding and operating device, the main structure of which is configured with a combination of multi-segment linkage shafts, so as to stably support the endoscopy for the doctor to easily operate the endoscopy. Furthermore, Republic of China Invention Patent No. 1674875 “Cutting and Clamping Device of Endoscopy” discloses an endoscopy clamping device with a multi-linkage structure, to enable the multi-angle adjustment of the endoscopy, thereby increasing the convenience of operation.

The conventional endoscopy holding device or surgical instrument holding device has following problem. In order to inspect and confirm the actual condition of the affected area, the doctor must repeatedly view the affected area in the body from different angles and directions during the operation. However, the conventional endoscopy holding device is unable to continuously fasten the endoscopy at the entry point, so the doctor inevitably disturbs, tears or even enlarges the incision during the repeated inspection of the affected area, and it causes secondary trauma and wound enlargement on the incision, slow postoperative wound healing, or even serious scar hyperplasia and ugly scar appearance.

For this reason, what is needed is to develop a new surgical instrument holder for medical purpose to hold various invasive surgical instruments including endoscopy, so as to prevent from secondary injury for incision during operation of the invasive surgical instrument.

Hence, there is a need to solve the above deficiencies/issues.

### SUMMARY

In order to solve the problems of conventional surgical instrument holder and especially prevent the secondary damage to the incision, the present invention proposes a new surgical instrument holder, which is able to hold various invasive surgical instruments and form a stagnation point, so that the surgical instrument is allowed to move freely while the invasive surgical instrument is stayed and positioned on the incision; therefore, during the operation, no matter how the doctor manipulates the surgical instrument, the surgical instrument holder in accordance with the present invention can prevent the surgical instrument from disturbing, tearing off, or even stretching the incision, so as to avoid the secondary damage to the incision.

Accordingly, the present invention provides a first surgical instrument holder, which includes an assembly of linkages including a first set of linkages and a second set of linkages; a securing module incorporated into the first set of linkages, configured to secure a surgical instrument and define a first line of center in parallel to the second set of linkages; a position indication module configured to provide a position indication line in parallel to parallel to the first line of center; and a complex lockable joint incorporated into the second set of linkages, including a turn piece to selectively lock up the assembly of linkages and define a second line of center in parallel to the first set of linkages, wherein where the first line of center and the second line of center intersect each other forms a stagnation point that is adjustable to stay and position at an entry point where the surgical instrument enters human body and positionally indicted by the position indication line for a user.

The present invention further provides a second surgical instrument holder, which includes an assembly of linkages comprising a first line of center and a second line of center, wherein where the first line of center and the second line of center intersect each other forms a stagnation point; a securing module configured to secure a surgical instrument and define a first line of center; and a complex lockable joint configured to define a second line of center, and comprising a turn piece to selectively lock up the assembly of linkages.

The above content described in the summary is intended to provide a simplified summary for the presently disclosed invention, so that readers are able to have an initial and basic understanding to the presently disclosed invention. The above content is not aimed to reveal or disclose a comprehensive and detailed description for the present invention, and is never intended to indicate essential elements in various embodiments in the present invention, or define the scope or coverage in the present invention.

### DESCRIPTION OF THE DRAWINGS

A more complete appreciation of the invention and many of the attendant advantages thereof are readily obtained as the same become better understood by reference to the following detailed description when considered in connection with the accompanying drawing, wherein:

FIG. 1 is an exploded view illustrating a main structure of a surgical instrument holder in accordance with the present invention;

FIG. 2 is an assembled view illustrating a holistically assembled structure of the surgical instrument holder in accordance with the present invention;

FIGS. 3A and 3B are schematic diagrams illustrating minimally-invasive nasal cavity surgery applying the surgical instrument holder in accordance with the present invention to hold an endoscopy;

FIG. 4 is a schematic diagram illustrating minimally invasive spinal surgery applying the surgical instrument holder in accordance with the present invention to clamp a working channel; and

FIG. 5 is a schematic diagram illustrating the surgical instrument holder in accordance with the present invention locked on the head fastening frame.

### DETAILED DESCRIPTION

The present disclosure will be described with respect to particular embodiments and with reference to certain drawings, but the disclosure is not limited thereto but is only limited by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice.

It is to be noticed that the term “including”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device including means A and B” should not be limited to devices consisting only of components A and B.

The disclosure will now be described by a detailed description of several embodiments. It is clear that other embodiments can be configured according to the knowledge of persons skilled in the art without departing from the true technical teaching of the present disclosure, the claimed disclosure being limited only by the terms of the appended claims.

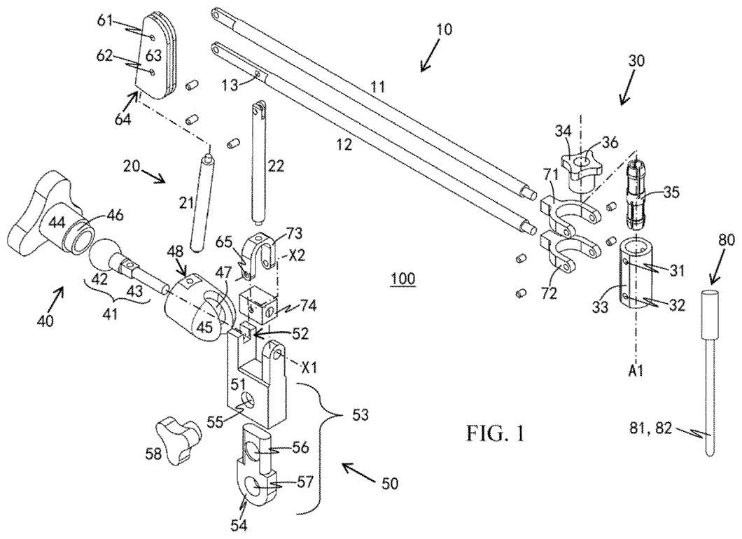

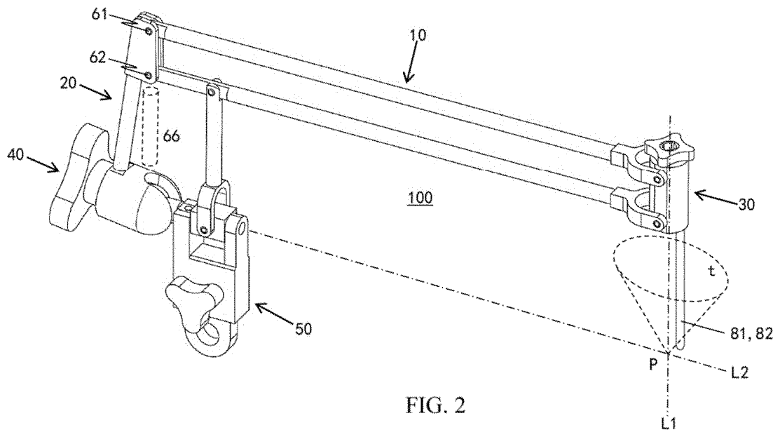

Please refer to FIGS. 1 and 2. FIG. 1 is an exploded view illustrating a main structure of a surgical instrument holder in accordance with the present invention, and FIG. 2 is an assembled view illustrating a holistically assembled structure of the surgical instrument holder in accordance with the present invention. A surgical instrument holder 100 in accordance with the present invention includes an assembly of linkages (main linkage mechanism), a securing module 30, a complex lockable joint 40, and a base 50. The assembly of linkages is the main linkage mechanism, and includes a first set of linkages 10 and a second set of linkages 20. The securing module 30 is disposed on an end of the first set of linkages 10, and the complex lockable joint 40 is disposed on an end of the second set of linkages 20 and pivotally connected to the pivot holder 52 of the base 50. The base 50 includes a connection part 53 configured to facilitate the surgical instrument holder 100 to further connect to an external mechanism such as a universal arm, a rail, a head fastening frame or a position adjusting mechanism. The external mechanism is used for primary adjustment for the position of the surgical instrument holder 100.

The first set of linkages 10 includes a first linkage 11 and a second linkage 12 disposed in parallel with each other, an end of the first linkage 11 and an end of the second linkage 12 are connected and secured to a first movable contact 61 and a second movable contact 62 of the multi-linkage connecting member 63, respectively; the other end of the first linkage 11 and the other end of the second linkage 12 are connected to the first Y-shaped connector 71 and the second Y-shaped connector 72, respectively, and are connected and secured to the third movable contact 31 and the fourth movable contact 32 of the securing module 30 through the first Y-shaped connector 71 and the second Y-shaped connector 72, respectively.

Preferably, the distance between the first movable contact 61 and the second movable contact 62 is equal to the distance between the third movable contact 31 and the fourth movable contact 32, and this structural configuration makes the first linkage 11 and the second linkage 12 parallel to each other, so that the first linkage 11, the second linkage 12, the multi-linkage connecting member 63 and the securing module 30 together form a quad parallel motion linkage structure, and the connection between centers of the third movable contact 31 and the fourth movable contact 32 forms a virtual first line of center L1.

Preferably, the complex lockable joint 40 is a ball-pivot lockable compound joint including a ball-type joint 41, a turn piece 44 and a directional ball-type bearing holder 45 which are assembled along an assembly axis X1. The ball-type joint 41 includes a ball body 42 and a pivot pin 43 connected to the ball body 42. Preferably, the ball body 42 and the pivot pin 43 are in an integral structure to form the ball-type joint 41. The directional ball-type bearing holder 45 has a 180-degree curved opening 47 configured to provide the pivot pin 43 to pass therethrough, and the ball-type joint 41 is disposed on the directional ball-type bearing holder 45 by using the directional ball-type bearing holder 45 as a bearing, so as to form a spherical pair of a kinematic pair.

The turn piece 44 includes an outer thread 46, the directional ball-type bearing holder 45 includes an inner thread, and the outer thread 46 and the inner thread are screwed correspondingly to form a helical pair. The pivot pin 43 of the ball-type joint 41 is inserted through the curved opening 47 of the directional ball-type bearing holder 45, so that the ball body 42 is fastened in the holder inside the directional ball-type bearing holder 45, and the pivot pin 43 is fastened in the pivot holder 52 of the base 50 along the assembly axis X1 and pivotally secured in the pivot holder 52. Therefore, the outer thread 46 of the turn piece 44 and the inner thread of the directional ball-type bearing holder 45 can be aligned and locked tightly manually to make the ball-type joint 41 in the stop status.

The second set of linkages 20 includes a third linkage 21 and a fourth linkage 22, an end of the third linkage 21 is connected to the second securing connector 48 of the directional ball-type bearing holder 45 on the complex lockable joint 40, the other end of the third linkage is connected to the first securing connector 64 of the multi-linkage connecting member; an end of the fourth linkage 22 is connected to the third Y-shaped connector 7, and further connected to and secured on the connection module 74 through the sixth movable contact 65 of the third Y-shaped connector 73. The third Y-shaped connector 73 is connected to and movably secured on the connection module 74 along the assembly axis X2 and through the sixth movable contact 65, and the connection module 74 is pivotably secured on the pivot pin 43 along the assembly axis X1 by using the pivot pin 43 as a shaft. The other end of the fourth linkage 22 is connected to the fifth movable contact 13 of the second linkage 12.

The first movable contact 61, the second movable contact 62 and the center of ball are substantially collinear on a virtual straight line, the connection between the second movable contact 62 and the center of ball can form a virtual linkage 66 which is substantially in parallel to the fourth linkage 22. The virtual linkage 66, the fourth linkage 22, the ball-type joint 41 and a part of the second linkage 12 together form a virtual quad parallel motion linkage structure. The connection between centers of the center of ball and the sixth movable contact 65, or the axis extended from the pivot pin 43 of the ball-type joint 41 form a virtual second line of center L2. The second line of center L2 and the first line of center L1 intersect at the stagnation point P.

The base 50 includes a main body 51; the connection part 53 of the base 50 includes a connection pin 54, a pin holder, a pin hole 55, a first connection hole 56, a second connection hole 57 and a manual bolt 58. The connection pin 54 and the pin holder of the main body 51 are complementary to and highly matched with each other in shape. The connection pin 54 is inserted into the pin holder, to make the first connection hole 56 align to the pin hole 55. The manual bolt 58 includes an outer thread, the pin hole 55 includes an inner thread, and the outer thread of the manual bolt 58 corresponds in thread to the inner thread of the pin hole 55. When the manual bolt 58 is inserted into the pin hole 55 and the outer thread of the manual bolt 58 is tightly locked with the inner thread of the pin hole 55, the connection pin 54 is secured on the main body 51 and the second connection hole 57 of the connection pin 54 is connected to the universal arm, the rail or corresponding component of other position adjusting mechanism. Therefore, the surgical instrument holder 100 in accordance with the present invention can be connected to and secured on an external position adjusting mechanism through the main body 51.

The securing module 30 includes an outer cylinder 33, an axial pressing piece 34 and an elastic collet 35. The outer cylinder 33 is substantively a short tube structure, and includes a third movable contact 31 and a fourth movable contact 32 disposed thereon. The outer cylinder 33, the axial pressing piece 34, and the elastic collet 35 are assembled along the same central axis A1. The axial pressing piece 34 has a round hole 36 form at where the central axis A1 passes. The axial pressing piece 34 includes an outer thread and the outer cylinder 33 includes an inner thread, so that the axial pressing piece 34 and the outer cylinder 33 can be screwed with each other.

The elastic collet 35 is a conical radial elastic collet, and the collet has a hollow clamping channel formed at a central part thereof, the hollow clamping channel corresponds in position to the round hole 36, so that the surgical instrument or the structure thereof can pass the hollow clamping channel and the round hole 36. When the axial pressing piece 34 and the outer cylinder 33 are being screwed with each other, the elastic collet 35 is axially pushed by the axial pressing piece 34 to generate a radial clamping force to clamp the surgical instrument or the structure thereof in the hollow clamping channel of the elastic collet 35. After the axial pressing piece 34 is screwed tightly, the surgical instrument or the structure thereof is secured on the securing module 30. Preferably, the surgical instrument is an endoscopy or a working channel.

When the base 50 is secured and the turn piece 44 of the complex lockable joint 40 is not locked tightly yet in an unlocked state, the first set of linkages 10, the second set of linkages 20, the securing module 30, the complex lockable joint 40 and components thereof are movable freely, but under mutual constraint between the first set of linkages 10 and the second set of linkages 20, no matter how the first set of linkages 10, the second set of linkages 20, the securing module 30, the complex lockable joint 40 and the components thereof are moved, the spatial position of the stagnation point P constantly stays at a certain specific point as long as the position of the base 50 is secured. Preferably, the stagnation point P is stayed and located at the specific entry point where the surgical instrument enters the human body.

The position indication module 80 is attached to and affixed on the securing module 30. The position indication module 80 preferably and at least includes a position indication bar 81, which provides a position indication line 82 that is in substantively parallel to the virtual first line of center L1 and approximately or roughly points toward the stagnation point P. The position indication line 82 is capable of roughly indicating the position of the stagnation point P for surgeon, to assist surgeon to manage the position of the stagnation point P and keep the surgical instrument staying in proximity to the entry point of human body. The position indication module 80 is selected from a position indication bar, an optical position indicator and a laser based position indicator.

For example, as shown in FIG. 2, a motion trace t of the securing module 30 is, for example but not limited to, elliptic or irregular, and the motion trace of the surgical instrument clamped by the securing module 30 can be conical. No matter what shape the trajectory t of the securing module 30 takes, as long as the base 50 is secured, the spatial position of the stagnation point P stays the same even if the turn piece 44 of the complex lockable joint 40 is not be tightly locked.

For example, when the stagnation point P is set to align to the entry point where the surgical instrument enters a human body and the surgical instrument connected to the securing module 30 is, for example but not limited to, the endoscopy, the securing module 30 is movable freely and the spatial position of the stagnation point P stays the same, so the endoscopy is prevented from disturbing, tearing off or even sketch the incision during the process in which the doctor repeatedly checks the condition of the internal organs and tissues of the human body, thereby preventing problems of causing secondary damage to the incision, enlarging wound, and causing slower wound healing, serious scar proliferation, and ugly scar appearance after surgery.

Once the doctor confirms the lesion, the doctor can rotate the turn piece 44 to tightly lock the complex lockable joint 40, and all movable components of the surgical instrument holder 100 are locked at once to make entire surgical instrument holder 100 enter the stop status or locked status; at this time, the spatial position of the stagnation point P is fixed, and entire surgical instrument holder 100 is highly rigid because of being in the locked status, so that the surgical instrument holder 100 can stably support the surgical instrument during the surgery.

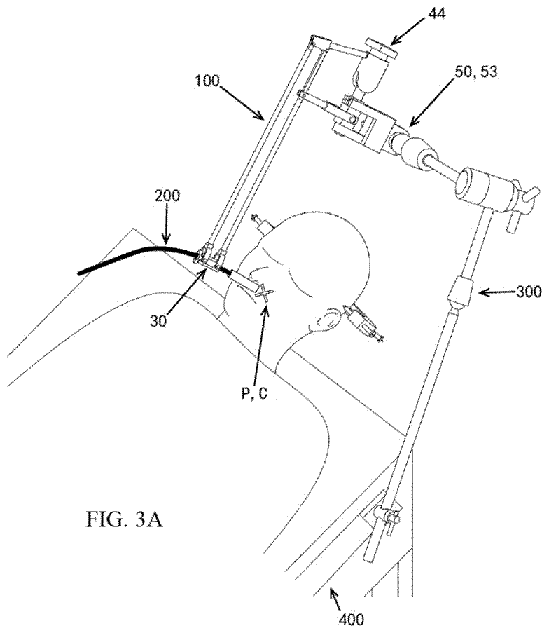

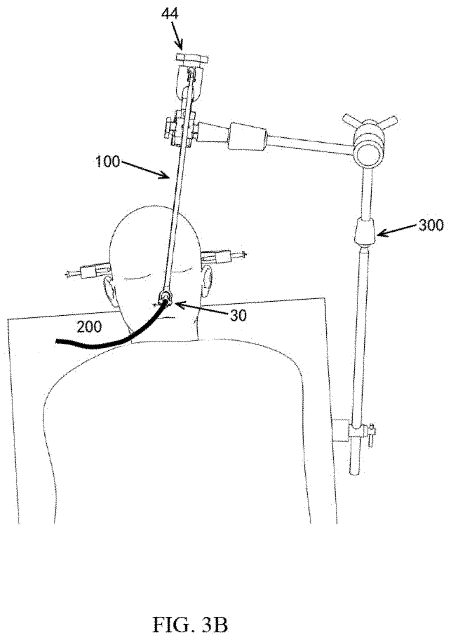

FIGS. 3A and 3B are schematic diagrams illustrating minimally-invasive nasal cavity surgery applying the surgical instrument holder in accordance with the present invention to hold an endoscopy. As shown in FIGS. 3A and 3B, the surgical instrument holder 100 in accordance with the present invention is connected to a medical universal arm 300 through the connection part 53 of the base 50, and a hose endoscopy 200 is clamped by the securing module 30. After the medical universal arm 300 is locked, the spatial position of the base 50 is determined, so that the spatial position of the stagnation point P is determined. Preferably, the spatial position of the stagnation point P is set as the spatial position of the entry point C, so that the doctor can operate the endoscopy 200 freely to check the condition of the lesion inside the nasal cavity as long as the turn piece 44 is not lock tightly in a loosened status.

Since the spatial position of the stagnation point P stays at the entry point C continuously, the endoscopy does not leave the entry point C even being rotated during the process where the doctor operate the endoscopy 200 to repeatedly check the inside of nasal cavity, thereby preventing from disturbing, tearing off or even enlarging the incision on the patient's body, and preventing the problems of causing the secondary wound to the incision, enlarging the wound, and causing slower wound healing, serious scar proliferation and ugly scar appearance after surgery. When the doctor's examination is completed, the doctor can just tightly lock the turn piece 44 to lock entire surgical instrument holder 100 at once, so that the surgical instrument holder 100 can stably support the endoscopy 200 during surgery.

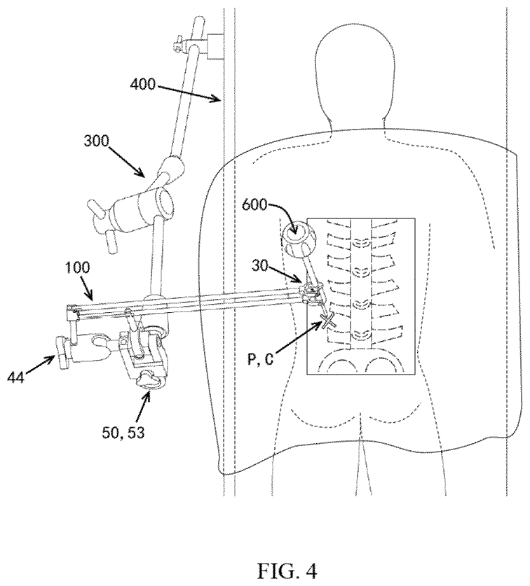

FIG. 4 is a schematic diagram illustrating minimally invasive spinal surgery applying the surgical instrument holder in accordance with the present invention to clamp a working channel. As shown in FIG. 4, the surgical instrument holder 100 in accordance with the present invention is connected to a linear rail 400 disposed on an edge of an operating bed or an operating table, through the connection part 53 of the base 50. A straight working channel 600 is clamped by the securing module 30. After the base 50 is locked up on the linear rail 400, the position of the base 50 is determined, so that the position of the stagnation point P is determined. Preferably, the position of the stagnation point P is set as the position of the entry point C, so that the doctor can operate the working channel 600 freely, treat, observe, examine, or perform surgery on the patient as long as the turn piece 44 is not lock tightly in the unlocked status.

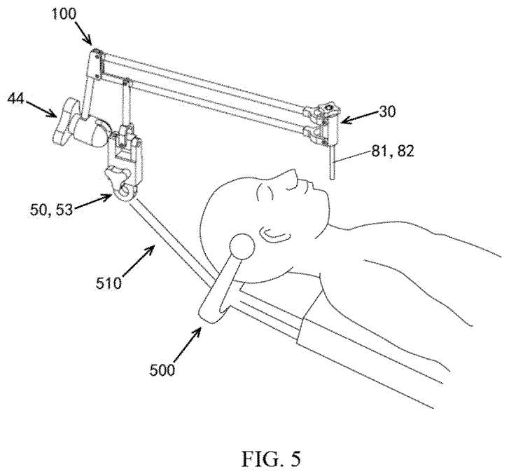

FIG. 5 is a schematic diagram illustrating the surgical instrument holder in accordance with the present invention locked on the head fastening frame. As shown in FIG. 5, the surgical instrument holder 100 in accordance with the present invention is connected to a relay linkage 510 through the connection part 53 of the base 50, and further connected to the head fastening frame 500 through the relay linkage 510. The securing module 30 is used to clamp any kind of surgical instrument. The position indication line 82 provided by the position indication bar 81 is capable of roughly indicating the position of the stagnation point P for surgeon to confirm the position of the stagnation point P. After the base 50 is locked up on the head fastening frame 500 and the position of the base 50 is determined, the doctor can adjust the position of the stagnation point P freely to operate the surgical instrument to perform surgery as long as the turn piece 44 is not lock tightly in the unlocked status.

According to the surgical instrument holder 100 in accordance with the present invention, the first set of linkages 10 and the second set of linkages 20 substantively form a dual parallel motion linkage as the main linkage mechanism, each of the two sets of parallel motion linkages is in parallel to an imaginary edge, and the two imaginary edges intersects at the crossover point P. When the compound joint is in the unlocked status, the endoscope clamper is movable freely, but the position of the crossover point P is fixed under the mutual constraint between the two sets of parallel motion linkages no matter how the two sets of parallel motion linkages are moved. With configuration of the compound joint, the two sets of parallel motion linkages are pivotal with the compound joint, and the position of the crossover point P constantly stays at a certain point no matter how the two sets of parallel motion linkages is pivoted.

The main linkage mechanism is mounted with a ball-pivot lockable compound joint at an end thereof, and the end of the main linkage mechanism is provided with a connection point configured to secure with the base; for example, the connection point can be connected to the rail on two sides of the operating bed, or secured on the universal arm of the rail, so as to increase the degree of freedom or space range of use. The compound joint is attached with a turn piece. The turn piece can be used to lock the compound joint and directly lock the entire endoscope clamper, so as to provide stable support for the endoscopy. The present invention proposes to use a single turn piece to directly lock the compound joint and the entire endoscope clamper.

The main linkage mechanism includes a clamping module mounted on other end thereof, and the clamping module is formed by the collet and the pressing mechanism and configured to clamp the endoscopy or other tubular structure, so that the user can adjust the clamping force (tightness) freely through the pressing mechanism. When the complex lockable joint is not locked up, the two sets of parallel motion linkages can perform a conical free rotation about the crossover point P, which serves as a top point of the cone, and the crossover point P is also called a rotation point. In the present invention, the rotation point constantly stays at the entry point of the surgical instrument, to prevent the endoscopy from repeatedly disturbing, tearing off, or even enlarging incision during operation process, and further prevent the problem of causing secondary damage to the incision, enlarging the wound, causing slow postoperative wound healing, more serious scar hyperplasia and ugly scar appearance after surgery.

There are further embodiments provided as follows.

Embodiment 1: A surgical instrument holder includes an assembly of linkages including a first set of linkages and a second set of linkages; a securing module incorporated into the first set of linkages, configured to secure a surgical instrument and define a first line of center in parallel to the second set of linkages; a position indication module configured to provide a position indication line in parallel to parallel to the first line of center; and a complex lockable joint incorporated into the second set of linkages, including a turn piece to selectively lock up the assembly of linkages and define a second line of center in parallel to the first set of linkages, wherein where the first line of center and the second line of center intersect each other forms a stagnation point that is adjustable to stay and position at an entry point where the surgical instrument enters human body and positionally indicted by the position indication line for a user.

Embodiment 2: The surgical instrument holder as described in Embodiment 1, further includes a base including: a pivot holder configured to movably connect to the complex lockable joint; a connection part configured to connect a universal arm, a rail, a head fastening frame or a position adjusting mechanism, so as to facilitate primary adjustment for a position of the stagnation point.

Embodiment 3: The surgical instrument holder as described in Embodiment 1, the first set of linkages further includes a first linkage and a second linkage arranged in parallel to each other; a multi-linkage connecting member including a first movable contact, a second movable contact, and a first securing connector; the securing module including a third movable contact and a fourth movable contact; wherein an end of the first linkage and an end of the second linkage are movably connected to the multi-linkage connecting member through the first movable contact and the second movable contact, respectively, and other ends of the first linkage and an end of the second linkage are movably connected to the securing module through the third movable contact and the fourth movable contact, respectively, so as to form a parallel structure with each other.

Embodiment 4: The surgical instrument holder as described in Embodiment 1, the complex lockable joint further includes a ball-type joint including a ball body and a pivot pin connected to the ball body; a directional ball-type bearing holder including a curved opening and a second securing connector, wherein the ball-type joint is disposed on the directional ball-type bearing holder by using the directional ball-type bearing holder as a bearing; wherein an interconnection among the first movable contact, the second movable contact and a center of ball of the ball body forms a virtual linkage.

Embodiment 5: The surgical instrument holder as described in Embodiment 1, the second set of linkages further includes a third linkage having an end connected to the first securing connector of the multi-linkage connecting member, and other end connected to the second securing connector of the complex lockable joint; a fourth linkage having an end connected to the second linkage through a fifth movable contact, and other end connected to a connection module through a sixth movable contact, wherein the connection module is pivotally secured with the pivot pin of the complex lockable joint; wherein the virtual linkage and the fourth linkage are in parallel to each other.

Embodiment 6: The surgical instrument holder as described in Embodiment 1, the securing module further includes an outer cylinder being a short tube structure which has a central axis and is configured to accommodate a pressing piece and an elastic collet, wherein the third movable contact and the fourth movable contact are disposed on the outer cylinder, wherein the pressing piece is configured to screw with the outer cylinder, the elastic collet generates a radial clamping force when being axially pushed by the pressing piece, so as to clamp the surgical instrument.

Embodiment 7: The surgical instrument holder as described in Embodiments 5 or 6, the first line of center is in parallel with the interconnection between the third movable contact and the fourth movable contact, the first line of center is in parallel to the central axis, the first line of center is in parallel to the fourth linkage, and the first line of center is in parallel to the virtual linkage.

Embodiment 8: The surgical instrument holder as described in Embodiments 4 or 5, the second line of center is in parallel to the interconnection between the center of ball and the sixth movable contact, the second line of center is in parallel to the first linkage, and the second line of center is in parallel to the second linkage.

Embodiment 9: The surgical instrument holder as described in Embodiment 1, the surgical instrument holder renders the stagnation point stay and position at an entry point where the surgical instrument enters human body, while the assembly of linkages is movable freely by using the stagnation point as a top point.

Embodiment 10: The surgical instrument holder as described in Embodiment 1, the position indication module is attached and affixed on the securing module, and is selected from a position indication bar, an optical position indicator and a laser based position indicator.

While the disclosure has been described in terms of what are presently considered to be the most practical and preferred embodiments, it is to be understood that the disclosure need not be limited to the disclosed embodiments. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present disclosure which is defined by the appended claims.

1. A surgical instrument holder, comprising:
an assembly of linkages comprising a first set of linkages and a second set of linkages;
a securing module incorporated into the first set of linkages, configured to secure a surgical instrument and define a first line of center in parallel to the second set of linkages;
a position indication module configured to provide a position indication line in parallel to parallel to the first line of center; and
a complex lockable joint incorporated into the second set of linkages, comprising a turn piece to selectively lock up the assembly of linkages and define a second line of center in parallel to the first set of linkages,
wherein where the first line of center and the second line of center intersect each other forms a stagnation point that is adjustable to stay and position at an entry point where the surgical instrument enters human body and positionally indicted by the position indication line for a user.

2. The surgical instrument holder as claimed in claim 1, further comprising:
a base comprising:
a pivot holder configured to movably connect to the complex lockable joint; and
a connection part configured to connect a universal arm, a rail, a head fastening frame or a position adjusting mechanism, so as to facilitate primary adjustment for a position of the stagnation point.

3. The surgical instrument holder as claimed in claim 1, wherein the first set of linkages further comprises:
a first linkage and a second linkage arranged in parallel to each other;
a multi-linkage connecting member comprising a first movable contact, a second movable contact, and a first securing connector; and
the securing module comprising a third movable contact and a fourth movable contact,
wherein an end of the first linkage and an end of the second linkage are movably connected to the multi-linkage connecting member through the first movable contact and the second movable contact, respectively, and other ends of the first linkage and an end of the second linkage are movably connected to the securing module through the third movable contact and the fourth movable contact, respectively, so as to form a parallel structure with each other.

4. The surgical instrument holder as claimed in claim 1, wherein the complex lockable joint further comprises:
a ball-type joint comprising a ball body and a pivot pin connected to the ball body; and
a directional ball-type bearing holder comprising a curved opening and a second securing connector, wherein the ball-type joint is disposed on the directional ball-type bearing holder by using the directional ball-type bearing holder as a bearing, wherein an interconnection among the first movable contact, the second movable contact and a center of ball of the ball body forms a virtual linkage.

5. The surgical instrument holder as claimed in claim 1, wherein the second set of linkages further comprises:
a third linkage having an end connected to the first securing connector of the multi-linkage connecting member, and other end connected to the second securing connector of the complex lockable joint; and
a fourth linkage having an end connected to the second linkage through a fifth movable contact, and other end connected to a connection module through a sixth movable contact, wherein the connection module is pivotally secured with the pivot pin of the complex lockable joint,
wherein the virtual linkage and the fourth linkage are in parallel to each other.

6. The surgical instrument holder as claimed in claim 1, wherein the securing module further comprises:
an outer cylinder being a short tube structure which has a central axis and is configured to accommodate a pressing piece and an elastic collet, wherein the third movable contact and the fourth movable contact are disposed on the outer cylinder,
wherein the pressing piece is configured to screw with the outer cylinder, the elastic collet generates a radial clamping force when being axially pushed by the pressing piece, so as to clamp the surgical instrument.

7. The surgical instrument holder as claimed in claim 5, wherein the first line of center is in parallel with the interconnection between the third movable contact and the fourth movable contact, the first line of center is in parallel to the central axis, the first line of center is in parallel to the fourth linkage, and the first line of center is in parallel to the virtual linkage.

8. The surgical instrument holder as claimed in claim 4, wherein the second line of center is in parallel to the interconnection between the center of ball and the sixth movable contact, the second line of center is in parallel to the first linkage, and the second line of center is in parallel to the second linkage.

9. The surgical instrument holder as claimed in claim 1, wherein the surgical instrument holder makes the stagnation point stay and position at an entry point where the surgical instrument enters human body, while the assembly of linkages is movable freely by using the stagnation point as a top point.

10. The surgical instrument holder as claimed in claim 1, wherein the position indication module is attached and affixed on the securing module, and is selected from a position indication bar, an optical position indicator and a laser based position indicator.

11. The surgical instrument holder as claimed in claim 6, wherein the first line of center is in parallel with the interconnection between the third movable contact and the fourth movable contact, the first line of center is in parallel to the central axis, the first line of center is in parallel to the fourth linkage, and the first line of center is in parallel to the virtual linkage.

12. The surgical instrument holder as claimed in claim 5, wherein the second line of center is in parallel to the interconnection between the center of ball and the sixth movable contact, the second line of center is in parallel to the first linkage, and the second line of center is in parallel to the second linkage.
